(19) United States
(12) Patent Application Publication
(10) Pub. No.: US 2025/0113852 A1
(43) Pub. Date: Apr. 10, 2025

(54) HUMAN FOOD PRODUCT

(71) Applicant: FermBiotics Holding APS, Baekke (DK)

(72) Inventor: Soren Kjaerulff, Holte (DK)

(73) Assignee: FermBiotics Holding APS, Baekke (DK)

(21) Appl. No.: 18/979,050

(22) Filed: Dec. 12, 2024

Related U.S. Application Data
(62) Division of application No. 17/269,172, filed on Feb. 17, 2021.

(30) Foreign Application Priority Data
Aug. 20, 2018 (DK) PA 2018 00479

(51) Int. Cl. A23L 33/135; A61K 35/744

(52) U.S. Cl. CPC A23L 33/135; A61K 35/744

(57) ABSTRACT

The present invention relates to a human food comprising a basic food product and an ingredient, wherein the ingredient comprising one or more health-enhancing microorganism and a combination of at least one fermented seaweed material and at least one fermented plant material.

### RELATED APPLICATIONS

This application is a divisional application of U.S. application Ser. No. 17/269,172, filed on Feb. 17, 2021, which is a national stage filing under 35 U.S.C. § 371 of International Application No. PCT/EP2019/072163, filed Aug. 19, 2019, which claims priority to Denmark Application No. PA 2018 00479, filed Aug. 20, 2018, each of which is incorporated by reference herein in its entirety.

### TECHNICAL FIELD OF THE INVENTION

The present invention relates to an improved human food product. In particular the present invention relates to a human food product capable of improving the intestinal adsorption from the human food product eaten.

### BACKGROUND OF THE INVENTION

In the modern society there is an increased interest and increased awareness on the nutritional value of the food products digested by humans. Increasing absorption of the nutrition from the food products consumed may lead to a need to consume smaller portions of food products, which may assist in the struggle people have with obesity.

Irrespective of the food product, optimising the nutritional value and the adsorption of food nutrients is a daily concern for all people, from infants to old people, for overweight people to thin people or even malnourished people, and athletes (irrespective of the athletes being armatures or professionals).

In respect of older people, it is known that by age, people become less active, their metabolism slows, their energy requirement decreases, all of which mean that they need to eat less, however, they would still need a certain level of nutrition adsorbed from the food digested.

With age, it is often observed that the dietary needs and uptake of nutrients from the food products digested change for several reasons, and older adults' abilities to absorb and utilize many nutrients become less efficient, their nutrient requirements (particularly as a function of body mass) actually increase. Furthermore, aging is often accompanied by a loss of appetite and changes in taste and smell, all of which can lead to more limited food choices and lower intake of healthful foods;

As with older adults, chronic conditions and medications may also affect nutrition requirements and special attention may be required. For example, in addition to drug-nutrient interactions affecting drug metabolism, some drug-nutrient interactions are also nutrient wasting and special required.

Due to these changes observed with older people, separate food product recommendations for people age 70 are made which may also be the case for athletes.

Hence, an improved human food product improving the nutritional adsorption by the human body would be advantageous, and in particular an improved human food product providing an improved nutritional adsorption by the human body, without health or physical side effects would be advantageous.

### SUMMARY OF THE INVENTION

Thus, an object of the present invention relates to an improved human food product that is capable of improving the nutritional adsorption.

In particular, it is an object of the present invention to provide an improved human food product, that is capable of improving the nutritional adsorption, that solves the above-mentioned problems of the prior art with reduced intestinal nutritional adsorption from the human food product eaten.

Thus, one aspect of the present invention relates to a human food comprising a basic food product and an ingredient, wherein the ingredient comprising one or more health-enhancing microorganism and a combination of at least one fermented seaweed material and at least one fermented plant material.

Another aspect of the present invention relates to a human food comprising a basic food product and an ingredient, wherein the ingredient comprising one or more health-enhancing microorganism and:
(i) at least one fermented seaweed material;
(ii) at least one fermented plant material; or
(iii) a combination of at least one fermented seaweed material and at least one fermented plant material,
   wherein the basic food product is selected the group consisting of a dairy product, a cereal product, a prepared food product; a confectionary product, a sport nutritional product and a beverage.

Yet an aspect of the present invention relates to use of the human food product according to the present invention for increasing the nutritional effect and/or nutritional adsorption of a human food product consumed by the human.

The present invention will now be described in more detail in the following.

### BRIEF DESCRIPTION OF THE DRAWINGS

FIG. 1 is a table illustrating an improved feed conversion rate and improved nutritional uptake for piglets receiving animal feed product according to the present invention;

FIG. 2a is a chart illustrating development of villi height fore increasing concentrations of the ingredient in animal feed product;

FIG. 2b is a chart illustrating an increased villi height in piglets receiving animal feed according to the present invention;

FIG. 3a is a chart illustrating improved intestinal enterocyte height development for piglets fed with an animal feed product supplemented with ingredients according to the present invention; and

FIG. 3b is a chart illustrating enterocyte height in piglets receiving animal feed according to the present invention.

### DETAILED DESCRIPTION OF THE INVENTION

Accordingly, as the nutritional benefits achieved by a consuming person is significantly decreasing the older the person gets and as people in general, in particular athletes are searching for optimising the food intake as well as the nutritional benefit, the human food industry is faced with a challenge in providing suitable human food products that is capable of improving the intestinal adsorption of the human food product consumed.

Surprisingly, the inventors of the present invention found an ingredient for a basic food product (providing a human food product), wherein the ingredient comprising one or more health-enhancing microorganism and: (i) at least one fermented seaweed material; (ii) at least one fermented plant material; or (iii) a combination of at least one fermented seaweed material and at least one fermented plant material, preferably, wherein the ingredient comprising a combination of at least one fermented seaweed material and at least one fermented plant material, is capable of improving the intestinal adsorption of the human food product consumed.

Furthermore, the inventors of the present invention found that improved nutritional effect of a human food product may be provided by a proper balance between digestibility and stimulation of intestinal microbiota and stimulation of intestinal mucosa. This improved nutritional effect is provided by the present invention.

A preferred embodiment of the present invention relates to a human food comprising a basic food product and an ingredient, wherein the ingredient comprising one or more health-enhancing microorganism and a combination of at least one fermented seaweed material and at least one fermented plant material.

Preferably, the basic food product may be selected the group consisting of a dairy product, a cereal product, a prepared food product; a confectionary product, a sport nutritional product and a beverage.

In a further preferred embodiment of the present invention relates to a human food product comprising a basic food product and an ingredient, wherein the ingredient comprising one or more health-enhancing microorganism and:
(i) at least one fermented seaweed material;
(ii) at least one fermented plant material; or
(iii) a combination of at least one fermented seaweed material and at least one fermented plant material,
   wherein the basic food product is selected the group consisting of a dairy product, a cereal product, a prepared food product; a confectionary product, a sport nutritional product and a beverage.

In the context of the present invention, the term “human feed product” relates to a substance to be consumed by a human.

In the context of the present invention, the term “comprising”, which may be synonymous with the terms “including”, “containing” or “characterized by”, relates to an inclusive or open-ended listing of features and does not exclude additional, unrecited features or method steps. The term “comprising” leaves the claim open for the inclusion of unspecified ingredients even in major amounts.

In an embodiment of the present invention, the dairy product may be an infant formula, a yoghurt, a butter, or a cheese.

In the event the dairy product is an infant formula, the infant formula may either be a dried product that need to be solubilized or suspended in an aqueous phase, such as water or milk; or the infant formula may be a liquid product ready to drink.

In the present context the term “yoghurt” relates to a food produced by bacterial fermentation of milk, in particular cow's milk. The bacteria used to make yogurt are known as “yogurt cultures”. Fermentation of lactose by these bacteria produces lactic acid, which acts on milk protein to give yogurt its texture and characteristic tart flavor.

The yoghurt may preferably be a liquid product in the form of a drinking yoghurt or in the form of an edible yoghurt. Edible yoghurt may be natural yoghurt, flavoured yoghurt, skyr, gayo, creme fraiche, fromage frais, or junket.

Cereal is any edible components of the grains. In an embodiment of the present invention the cereal product may be a grain product, a Müesli product, a raw grain product or a treated flour product (such as flour, or mixed flour products).

In an embodiment the human food product is a combined yoghurt product and cereal product. In such combined product (yoghurt and cereal) the ingredient according to the present invention may be supplied in the yogurt product or in the cereal product or both in the yoghurt and the cereal product.

In a further embodiment of the present invention the confectionary product may be a bakery confectionary product or a sugar confectionary product. Preferably, the bakery confectionary product is bread, pies, cakes, sweet pastries, doughnuts, scones, cookies, or chips (e.g. potato chips or corn chips).

Preferably, the sugar confectionary product may be sugar candies, chocolates, chewing gum, or ice cream.

In another embodiment of the present invention, the prepared food product may be a baby food, prepared dishes, mayonnaise, dressing, seasoning, dried seasoning powder, and various moss food products, or pesto.

Prepared food may relate to food which has been prepared by manufacturers and is sold to be eaten at home.

In an embodiment of the present invention the sport nutritional product may be a sport energy gel, a sport energy powder or a sport energy bar, a sport energy tablet, or a sport energy drink.

The optimal sport nutritional product may be the sport nutritional product in combination with a protein source, such as whey protein powder. Thus, in in embodiment of the present invention the sport nutritional product may further comprises a protein component, e.g. a whey protein powder.

The advantages of such a combined sport nutritional product is that people exhausted from sports have a reduced immune response and is more exposed to infections. The fermented product of the present invention has shown to increased nutritional uptake and provide improved inflammatory conditions and antimicrobial activities; whereas the protein component, e.g. whey protein powder, has been demonstrated to assist in building or re-building muscles and assisting in recovery from strong exercise.

In a preferred embodiment of the present invention the ingredient may consisting essentially of one or more health-enhancing microorganism and a combination of at least one fermented seaweed material and at least one fermented plant material.

In a further preferred embodiment of the present invention the ingredient may consisting essentially of one or more health-enhancing microorganism and:
(i) at least one fermented seaweed material;
(ii) at least one fermented plant material; or
(iii) a combination of at least one fermented seaweed material and at least one fermented plant material.

In the context of the present invention, the term “consisting essentially of”, relates to a limitation of the scope of a claim to the specified features or steps and those features or steps, not mentioned and that do not materially affect the basic and novel characteristic(s) of the claimed invention.

In an embodiment of the present invention the ingredient may represent in the range of 0.5-50% (w/w) of the human food product, such as in the range of 1-45% (w/w), e.g. in the range of 2-40% (w/w), such as in the range of 3-35% (w/w), e.g. in the range of 4-30% (w/w), such as in the range of 5-25% (w/w), e.g. in the range of 7.5-20% (w/w), such as in the range of 10-15% (w/w).

The human food product according to the present invention comprises the combination of a basic feed product and an ingredient.

Preferably, the basic food product may be a dairy product, a cereal product, a prepared food product; a confectionary product, a sport nutritional product and a beverage fodder product suitable for human consumption and without the presence of the ingredient of the present invention.

The term “at least one fermented seaweed material” implies that different fermented seaweed materials may be used. In an embodiment of the present invention the human feed product comprises at least one seaweed material, such as at least two seaweed materials, e.g. at least three seaweed materials, such as at least four seaweed materials.

In an embodiment of the present invention wherein the at least one fermented seaweed material may be a unicellular alga or a multicellular macroalgae.

In a further embodiment of the present invention the multicellular macroalgae may be selected from brown macroalgae, red macroalgae, and/or green macroalgae.

In another embodiment of the present invention the brown macroalgae may be selected from one or more of kelps, Saccharina latissimi (Laminaria saccharina), Laminaria digitate, Ascophyllum nodosum, Laminaria hyperborean, or a mixture hereof.

In the present context, the term “plant material” relates to land plant material capable of performing photosynthesis, excluding algae and fungi.

The term “at least one fermented plant material” implies that different plant materials may be used. In an embodiment of the present invention the animal feed product comprises at least one fermented plant material, such as at least two fermented plant materials, e.g. at least three fermented plant materials, such as at least four fermented plant materials.

When the animal feed product comprises two or more fermented plant materials, the fermented plant materials may be of different origin.

In an embodiment of the present invention the fermented plant material may be selected from at least one proteinaceous plant material. The proteinaceous plant material may be a vegetable plant material, preferably, the vegetable plant material may be selected from eudicot plants, angiosperm plants, and/or rosid plants.

Preferably the proteinaceous plant material or the vegetable plant material may be selected from Brassicale plants.

In an embodiment of the present invention the Brassicale plants is selected from the Brassicaceae family or the Cruciferae family.

In a further embodiment of the present invention the Brassicaceae family or the Cruciferae family may be selected from at least one of a Brassica genus; sun flower; palm; soya, field beans, Lupins; or a combination hereof. Preferably, at least one Brassica genus may be selected from one or more species such as Brassica napus; Brassica oleracea; Brassica campestris; Brassica nigra; Sinapis alba (Brassica alba); Brassica juncea; Brassica rapa or mixtures hereof.

In yet an embodiment of the present invention the at least one Brassica genus may be selected from the group consisting of: including rape, rapeseed, canola, cabbage, broccoli, cauliflower, kale, Brussels sprouts, collard greens, savoy, kohlrabi, gai lan, white mustard, Indian mustard, Chinese mustard, and black mustard seed powder.

The human food product may comprise a combination of at least one fermented seaweed material as defined above, and at least one fermented plant material as defined above.

In the present context, the term “fermented” relates to a material (at least one plant material, at least one seaweed material or a combination of at least one plant material and at least one seaweed material) relates to a controlled metabolic process of the material(s) by adding a predetermined amount of fermenting microorganisms to the material allowing the microorganism and the material(s) to interact breaking down the material.

The ingredient and the human food product comprise one or more health-enhancing microorganism, preferably the health-enhancing microorganism may be a health-enhancing yeast and/or a health-enhancing bacterium, even more preferably the health-enhancing microorganism may be a health-enhancing bacterium.

The health-enhancing bacteria may comprise one or more probiotics. The one or more probiotics and/or the one or more health-enhancing microorganism may comprise at least one lactic acid bacterial strain.

In the context of the present invention, the term “probiotic” relates to live microorganisms that when administered in adequate amounts, confer a health benefit on the host.

In a preferred embodiment of the present invention the at least one lactic acid bacterial strain may be selected from the group consisting of the genus Enterococcus, Lactobacillus, Pediococcus, Lactococcus, or Bifidobacterium or combinations thereof.

In a further embodiment of the present invention the one or more lactic acid bacterial stain(s) may be selected from the group consisting of Pediococcus pentosaceus; Pendiococcus acidilactici; Lactobacillus plantarum, Lactobacillus rhamnosus, Enterococcus faecium, Lactobacillus acidophilus, Bifidobacterium lactis, Bifidobacterium longum, Bifidobacterium bifidum, Lactobacillus salivarius, Lactobacillus pentoses, Lactobacillus vaginalis, Lactobacillus xylosus and a combination thereof.

In an embodiment of the present invention the health-enhancing microorganism may be the main microorganism present in the human food product. Preferably, the main microorganism is a lactic acid bacterium. Even more preferably the main microorganism may be selected from the groups consisting of Pediococcus pentosaceus; Pendiococcus acidilactici, Lactobacillus plantarum, Lactobacillus rhamnosus, Enterococcus faecium, Lactobacillus acidophilus, Bifidobacterium lactis, Bifidobacterium longum, Bifidobacterium bifidum, Lactobacillus salivarius, Lactobacillus pentoses, Lactobacillus vaginalis, and Lactobacillus xylosus; preferably, the main lactic acid bacteria present in the composition is Lactobacillus plantarum.

In the context of the present invention the term “main microorganism” relates to the microorganism present in highest amount, determined on a weight/weight ratio relative to the total number of microorganism present.

During fermentation a group of microorganisms may be used for fermenting the plant material to provide a co-fermentation. The co-fermentation may be a mixture of different microorganisms (such as a mixture of yeasts, fungus, and/or bacteria) or a mixture of different bacteria. Preferably the co-fermentation comprises a mixture of different bacterial strains. In an embodiment of the present invention the animal feed product comprises one or more bacterial strains, e.g. two or more bacterial strains, such as three or more bacterial strains, e.g. four or more bacterial strains, such as 7 or more bacterial strains, e.g. 10 or more bacterial strains, such as 15 or more bacterial strains, e.g. 20 or more bacterial strains, such as 25 or more bacterial strains, e.g. 30 or more bacterial strains, such as 35 or more bacterial strains, e.g. 40 or more bacterial strains. Preferably, the bacterial strains may be one or more lactic acid bacterial strains.

In a further embodiment of the present invention the one or more lactic acid bacteria stain(s) may be selected from the group consisting of one or more of Pediococcus pentosaceus (DSM 12834); Pendiococcus acidilactici (DSM 16243); Lactobacillus plantarum (DSM 12837); Enterococcus faecium (NCIMB 30122); Lactobacillus rhamnosus (NCIMB 30121); Pediococcus pentosaceus HTS (LMG P-22549); Pendiococcus acidilactici (NCIMB 30086); and/or Lactobacillus plantarum LSI (NCIMB 30083).

Preferably, the one or more lactic acid bacteria strain(s) may be selected from the group consisting of one or more Pediococcus pentosaceus (DSM 12834), Pendiococcus acidilactici (DSM 16243) and/or Lactobacillus plantarum (DSM 12837). Preferably, the lactic acid bacteria strain(s) comprises the combination of Pediococcus pentosaceus (DSM 12834), Pendiococcus acidilactici (DSM 16243) and Lactobacillus plantarum (DSM 12837).

The human food product may have a high content of viable lactic acid bacteria. In an embodiment of the present invention the human food product comprises one or more lactic acid bacterial strain(s) in a total amount in the range of 10⁵-10¹² CFU per gram of the human food product, such as in the range of 10⁶-10¹² CFU per gram, e.g. in the range of 10⁷-10¹¹ CFU per gram, such as in the range of 10⁸-10¹¹ CFU per gram, e.g. in the range of 10⁹-10¹⁰ CFU per gram.

The ingredient may have a high content of viable lactic acid bacteria. In an embodiment of the present invention the ingredient comprises one or more lactic acid bacterial strain(s) in a total amount in the range of 10⁵-10¹² CFU per gram of the human food product, such as in the range of 10⁶-10¹² CFU per gram, e.g. in the range of 10⁷-10¹¹ CFU per gram, such as in the range of 10⁸-10¹¹ CFU per gram, e.g. in the range of 10⁹-10¹⁰ CFU per gram.

In an embodiment of the present invention the human food product may comprise a lactic acid concentration of at least 0.05% lactic acid relative to the human food product, such as at least 0.1% lactic acid relative to the human food product, such as at least 0.5% lactic acid relative to the human food product, e.g. at least 0.75% lactic acid relative to the human food product, such as at least 1% lactic acid relative to the human food product, e.g. at least 2.5% lactic acid relative to the human food product, at least 5% lactic acid relative to the human food product, such as in the range of 0.05-5% lactic acid relative to the human food product, such as in the range of 0.1-2.5% lactic acid relative to the human food product, such as in the range of 0.5-1% lactic acid relative to the human food product.

During fermentation, various metabolites may be formed, which in combination, may assist in the enhancing the nutritional and antibiotic benefits from using the ingredient according to the present invention in the human food product.

In an embodiment of the present invention the fermentation process for providing the at least one fermented plant material; the at least one fermented seaweed material; or the combination of the at least one fermented plant material and the at least one fermented seaweed material (fermented separately or jointly) may be performed for a period in the range of 24 hours to 30 days, such as for a period in the range of 2-25 days, e.g. for a period in the range of 5-20 days, such as for a period in the range of 7-15 days, e.g. for a period in the range of 10-13 days, e.g. about 11 days.

If fermentation is performed jointly with the plant material and the seaweed material, the fermentation of the seaweed material may be started first and the plant material may be added subsequently. In an embodiment of the present invention the seaweed material may be subjected to fermentation for 1-15 days before the plant material is added; such as 3-12; e.g. 5-10; such as about 7 days before the plant material is added. When the plant material is added to the fermented plant material the fermentation may be continued with both the seaweed and the plant material for 2-20 days; such as for 4-16 days; e.g. for 8-13 days; e.g. for about 11 days.

In a further embodiment of the present invention the fermentation process for providing the at least one fermented plant material; the at least one fermented seaweed material; or the combination of the at least one fermented plant material and the at least one fermented seaweed material (fermented separately or jointly) may be performed at a temperature in the range of 25-45° C.; such as in the range of 30-43° C.; e.g. in the range of 32-40° C., such as about 35° C.

During fermentation the pH of the material fermented may preferably drop within the first 24 hours of fermentation to pH 6 or below, such as pH 5 or below, e.g. pH 4.5 or below, such as pH 4.2 or below. Preferably, the drop in pH may be provided by the fermentation process, preferably no addition of acid may be made.

When the ingredient or the human food product comprises the combination of at least one fermented plant material and at least one fermented seaweed material, the fermentation of the at least one plant material and the fermentation of the at least one seaweed material may be performed separately or jointly. Preferably, the fermentation of the at least one plant material and the fermentation of the at least one seaweed material may be performed jointly.

The human food product according to the present invention may preferably comprises a fibrous material. Preferably the human food product comprises the fibrous material originating from the plant material and/or the seaweed material.

In an embodiment of the present invention the human food product comprises more than 5 g fibrous material per kg dried human food product, such as more than 10 g fibrous material per kg dried human food product, e.g. more than 15 g fibrous material per kg dried human food product, such as more than 20 g fibrous material per kg dried human food product, e.g. more than 25 g fibrous material per kg dried human food product, such as more than 50 g fibrous material per kg dried human food product, e.g. more than 75 g fibrous material per kg dried human food product, such as more than 100 g fibrous material per kg dried human food product, e.g. more than 150 g fibrous material per kg dried human food product, such as more than 200 g fibrous material per kg dried human food product, e.g. more than 250 g fibrous material per kg dried human food product, such as more than 300 g fibrous material per kg dried human food product.

The human food product according to the present invention may comprises a starch material. Preferably the human food product comprises the starch material originating from the plant material and/or the seaweed material.

In an embodiment of the present invention wherein the composition may comprise more than 5 g starch material per kg dried human food product, such as more than 10 g starch material per kg dried human food product, e.g. more than 15 g starch material per kg dried human food product, such as more than 20 g starch material per kg dried human food product, e.g. more than 25 g starch material per kg dried human food product, such as more than 50 g starch material per kg dried human food product, e.g. more than 75 g starch material per kg dried human food product, such as more than 100 g starch material per kg dried human food product, e.g. more than 150 g starch material per kg dried human food product, such as more than 200 g starch material per kg dried human food product, e.g. more than 250 g starch material per kg dried human food product, such as more than 300 g starch material per kg dried human food product.

It may be preferred that the human food product according to the present invention may be a dried composition. Preferably having a moisture content of 15% (w/w) or less, such as less than 12.5% (w/w), e.g. less than 10% (w/w), such as less than 7.5% (w/w), e.g. less than 5% (w/w).

In an embodiment of the present invention the at least one plant material and/or the at least one seaweed material may subjected to at least one pre-treatment of the at least one plant material and/or the at least one seaweed material resulting in the above mentioned average maximum diameter.

The at least one pre-treatment may involve disintegration, chopping, sheading, crushing, grinding, or the like of the at least one plant material and/or the at least one seaweed material, in order to reduce the size of the at least one plant material and/or the at least one seaweed material.

In an embodiment of the present invention the pre-treatment may be performed on at least one dried, fresh or frozen plant material and/or on at least one dried, fresh or frozen seaweed material.

The at least one plant material and/or the at least one seaweed material may have an average maximum diameter of 5 cm, such as an average maximum diameter of 4 cm such as an average maximum diameter of 3 cm, such as an average maximum diameter of 2 cm, such as an average maximum diameter of 1 cm, such as an average diameter in the range 25 um to 5 cm, such as 0.1 mm to 5 cm, such as an average diameter in the range of 0.5 mm to 5 cm, such as an average diameter in the range 0.5 mm to 2 cm.

The human food product according to the present invention may comprise a content of the ingredient in the range of 0.5-25% (w/w), such as in the range of 1-20% (w/w), e.g. in the range of 2-18% (w/w), such as in the range of 3-16% (w/w), e.g. in the range of 5-15% (w/w), such as in the range of 7-12% (w/w), e.g. in the range of 8-10% (w/w).

The fermented seaweed material and/or the fermented plant material, may preferably not be subjected to sterilisation in order to maintain the native metabolic nature of the materials.

As mentioned previously, the inventors of the present invention found that the best nutritional effect was obtained from providing a balance between digestibility of the fermented ingredient and stimulation of the intestinal microbiota and/or stimulation of the intestinal mucosa. In order to meet this balance different fractions of particle sizes of the fermented ingredient may be provided.

In an embodiment of the present invention the fermented ingredient may be a dried fermented ingredient. The drying of the fermented ingredient may preferably be a gentle drying method in order to maintain the active potential of the fermented ingredient. An example of a gentle drying method may be as described in WO 2013/029632.

The fermented ingredient may be mixed with the basic feed compound as a free-flowing powder or before animal feed pellets are formed, resulting in animal feed pellets comprising the fermented ingredient. Preferably the fermented ingredient is mixed with the basic feed compound as a dry fermented ingredient.

In an embodiment of the present invention the dried fermented ingredient comprises a range of 30-70% (w/w); such as a range of 40-60% (w/w); e.g. about 50% (w/w) of the dry fermented ingredient has a particle size below 0.5 mm and a range of 30-70% (w/w); such as a range of 40-60% (w/w); e.g. about 50% (w/w) of the dry fermented ingredient has a particle size above 0.5 mm.

In a further embodiment of the present invention the fermented ingredient may be a dried fermented ingredient, and wherein a range of 20-80% (w/w) of the dry fermented ingredient has a particle size below 0.5 mm (preferably in the range of 0.01-0.5 mm), and wherein 20-80% (w/w) of the dry fermented ingredient comprises a particle size above 0.5 mm (preferably in the range of 0.5-1.0 mm)

In an even further embodiment of the present invention the dried fermented ingredient comprises at least 2, preferably at least 3, even more preferably at least 4 of the following criteria:
a) 1-10% (w/w), such as about 5% (w/w), of the dry fermented ingredient has a particle size above 1.0 mm;
b) 45-55% (w/w), such as about 50% (w/w), of the dry fermented ingredient has a particle size between 0.5-1.0 mm;
c) 30-40% (w/w), such as about 50% (w/w), of the dry fermented ingredient has a particle size between 0.25-0.5 mm; and/or
d) 5-15% (w/w), such as about 10% (w/w), of the dry fermented ingredient has a particle size below 0.25 mm.

In the present context the term “about” relates to a variation on the stated amount of 10% or less, such as 5% or less, e.g. 1% or less.

The selection of the various particle sizes may be determined by sieving as known to the skilled person.

Conditions for providing and fermenting the fermented seaweed material; the fermented plant material, or the combination of fermented seaweed material and fermented plant material, may be as described in WO 2008/006382; WO 2014/206419; or PCT/EP2016/076952. WO 2008/006382; WO 2014/206419; or PCT/EP2016/076952 are hereby incorporated by reference.

The human food product according to the present invention may preferably induce an increased nutritional effect and/or nutritional adsorption of the human food product by a consuming human.

Thus, the human food product may be used in a method for improving or increasing the increased nutritional effect and/or increased nutritional adsorption of the human food product by a consuming human.

When food is consumed it becomes degraded through the digestive system and in the intestine nutrients from the food product may be absorbed into the body.

Intestinal villi are small, finger-like projections that extend into the lumen of the small intestine. Each villus has many microvilli projecting from the enterocytes of its epithelium which collectively form the striated or brush border.

Intestinal villi/microvilli increase the internal surface area of the intestinal walls making available a greater surface area for absorption of the nutrients from the digested food product. An increased absorptive area is useful because digested nutrients (including monosaccharide and amino acids) pass into the semipermeable villi through diffusion and becomes adsorbed by the body.

In an embodiment of the present invention, the human food product according to the present invention may increase the height/length of the intestinal villi by at least 5%, such as at least 10%, e.g. at least 15%, such as at least 20%, e.g. at least 25%, such as at least 30%, e.g. at least 35%, such as at least 40%, e.g. at least 45%, such as at least 50%, e.g. at least 60%, such as at least 75%.

In another embodiment of the present invention, the human food product according to the present invention may increase the height/length of the intestinal microvilli by at least 5%, such as at least 10%, e.g. at least 15%, such as at least 20%, e.g. at least 25%, such as at least 30%, e.g. at least 35%, such as at least 40%, e.g. at least 45%, such as at least 50%, e.g. at least 60%, such as at least 75%.

Not only the intestinal villi or intestinal microvilli are increased in height from eating the human food product according to the present invention, but also the intestinal enterocytes are increased in height.

In an embodiment of the present invention, the human food product according to the present invention may increase the height/length of the intestinal enterocytes by at least 5%, such as at least 10%, e.g. at least 15%, such as at least 20%, e.g. at least 25%, such as at least 30%, e.g. at least 35%, such as at least 40%, e.g. at least 45%, such as at least 50%, e.g. at least 60%, such as at least 75%.

In a further embodiment of the present invention a method is provided for improving the feed conversion rate of a food product in a human, wherein the method comprises the step of administering a human food product according to the present invention to a human.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

### Example 1

Determining the effect of the animal feed product according to the present invention on feed conversion rate, nutritional uptake and incidence of diarrhoea.

### Method

The experiments were conducted at a private, commercial farm and included:
1350 sows.
33.5 weaned piglets per sow annually.
440 gram per day on average daily gain in weaner unit (exit weight: ˜33 kg)
   Feeding was performed using 4 different dietary treatment groups (TG) all groups were mash feed.
Litters were standardized to 14 piglets per sow; fixed from day 14 post-partum.
Differential feeding
   start: 10 days before weaning; end: at exit from weaner unit.
The animal was followed until slaughter (˜115 kg).
   No systemic or individual antibiotics treatment of experimental pigs was done.
Piglets needing treatment were eliminated from the experiment and treated elsewhere.
   15 days after weaning, 6 piglets/TG were slaughtered and blood and intestines were analysed.

The following 4 different treatment groups (TG) each comprising 50 piglets were provided:
TG1: No supplementation (negative control)
TG2: 2500 ppm zinc oxide (positive control)
TG3: 8% of DM as prefermented rape seed
TG4: 10% of DM as prefermented rape seed

Supplements were mixed into the standard pre-weaner and starter diets.

Statistical analyses were performed including TG, gender and their interactions, start weight.

### Results

The results demonstrate that the feed according to the present invention resulted in an increased feed conversion rate, increased nutritional uptake and a reduced incidence of diarrhoea.

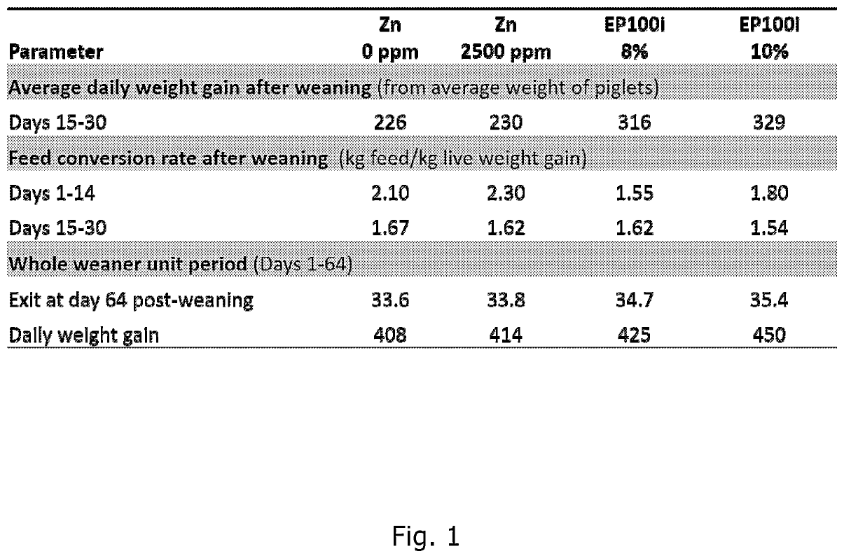

FIG. 1 illustrates an increased average daily weight gain after weaning for pigs of 15-30 days old piglets. The results show that by adding 8% fermented rape seed (EP100i) to the animal feed product or ingredient according to the present invention, the average daily weight gain after weaning for pigs of 15-30 days old piglets was increased 40% relative to feed products not supplemented with medicinal zinc and 37% relative to feed products supplemented with 2500 ppm medicinal zinc. FIG. 1 illustrate that increasing the concentration of fermented rape seed (EP100i) to 10% the average daily weight gain after weaning for pigs of 15-30 days old piglets was further increased to 46% relative to feed products not supplemented with medicinal zinc and 43% relative to feed products supplemented with 2500 ppm medicinal zinc.

It is further illustrated in FIG. 1, that the feed conversion rate after weaning (kg feed/kg live weight gain) was increased for piglets (divided in groups 1-14 days of age and 15-30 days of age) receiving the animal feed supplemented with 8% fermented rape seed according to the present invention. FIG. 1 show a significant increase in the feed conversion rate for piglets of 1-14 days of age 26% relative to feed products not supplemented with medicinal zinc and 33% relative to feed products supplemented with 2500 ppm medicinal zinc, whereas piglets of 15-30 days of age only an increase of 3% relative to feed products not supplemented with medicinal zinc was observed and no increased was observed relative to feed products supplemented with 2500 ppm medicinal zinc. Increasing the concentration of fermented rape seed (EP100i) to 10% the feed conversion rate for pigs of 1-14 days of age and 15-30 days of age, was also increased for both feed products not supplemented with medicinal zinc and for feed products supplemented with 2500 ppm medicinal zinc.

From FIG. 1 it is shown that an increased weight at exit from the weaner unit (91 days of age/64 post weaning) was approximately 1-1.7 kg higher for piglets receiving the animal feed product according to the present invention relative to feed products not supplemented with medicinal zinc and relative to feed products supplemented with 2500 ppm medicinal zinc, as well as an increased daily weight gain was observed for piglets receiving the animal feed product according to the present invention relative to feed products not supplemented with medicinal zinc and relative to feed products supplemented with 2500 ppm medicinal zinc.

Thus, FIG. 1 clearly illustrates an improved feed conversion rate and an improved nutritional uptake for piglets receiving the animal feed product according to the present invention relative to feed products not supplemented with medicinal zinc and relative to feed products supplemented with 2500 ppm medicinal zinc.

The improved feed conversion rate and the improved nutritional uptake for piglets may be a result of an increase in microvilli height and/or endocyte height.

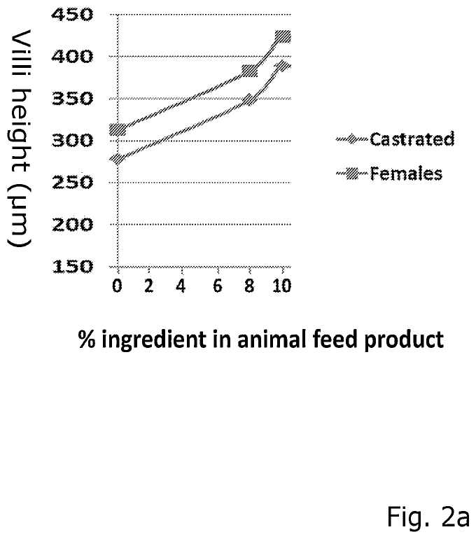

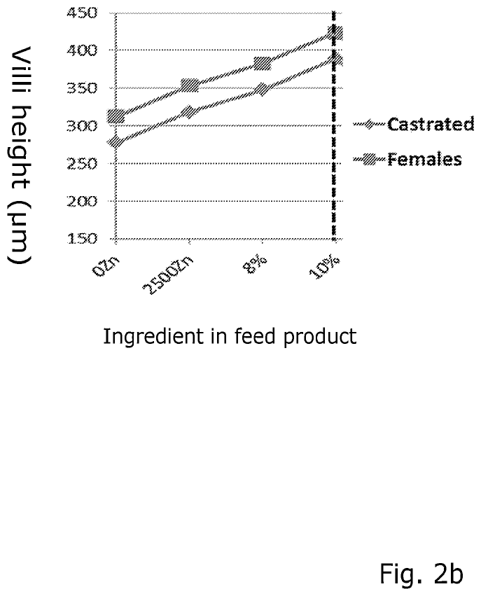

FIG. 2a illustrates the development of villi height, determined in um, for increasing concentrations of the ingredient in the animal feed product from 0 to 10% ingredient in the animal feed product. The ingredient was based on rapeseed which has been fermented using a mixture of Pediococcus pentosaceus (DSM 12834), Pendiococcus acidilactici (DSM 16243) and/or Lactobacillus plantarum (DSM 12837). FIG. 2b illustrate an increased villi height in piglets receiving animal feed according to the present invention relative to feed products not supplemented with medicinal zinc and relative to feed products supplemented with 2500 ppm medicinal zinc.

The results in FIGS. 2a and 2b shows that a significant increase in villi height is provided when increasing the amount of ingredient from 0% to 10% whereby the surface area of the intestine for absorbing nutrients are provided whereby the improved feed conversion rate and the improved nutritional uptake for piglets may be provided.

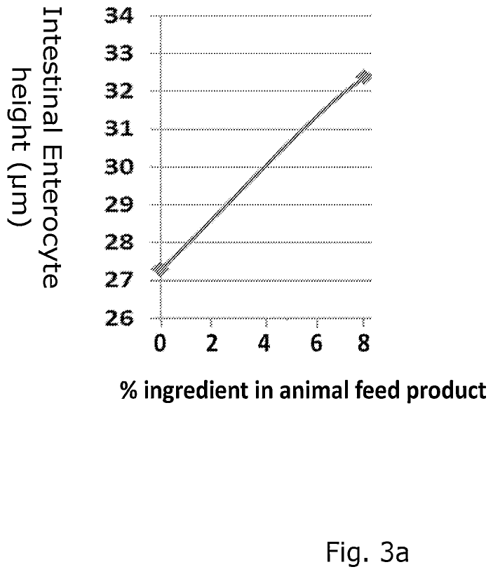

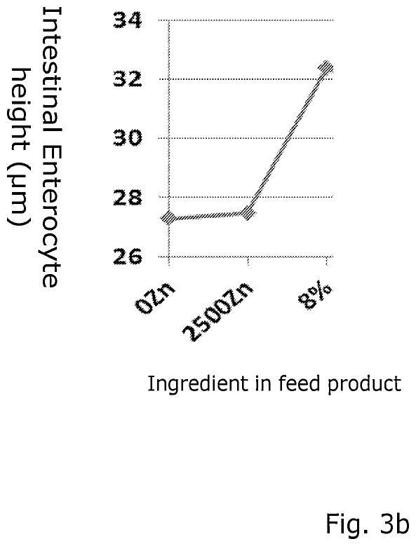

Also an improved intestinal enterocyte height development (determined in um) are demonstrated for piglets feed with an animal feed product supplemented with the ingredient according to the present invention, see FIG. 3a. The ingredient was based on rapeseed which has been fermented using a mixture of Pediococcus pentosaceus (DSM 12834), Pendiococcus acidilactici (DSM 16243) and/or Lactobacillus plantarum (DSM 12837)

The results presented in FIG. 3a show a significant increase in enterocyte height when increasing the concentration of the ingredient in the animal feed product from 0% to about 8%. FIG. 3b illustrate an increased enterocyte height in piglets receiving animal feed according to the present invention relative to feed products not supplemented with medicinal zinc and relative to feed products supplemented with 2500 ppm medicinal zinc.

Analysing the piglet diarrhoea after weaning showed a reduced incidence of diarrhoea days and shorter duration of diarrhoea infection for piglets receiving the animal feed product according to the present invention, comparted not only to the group not receiving any Zn but also relative to the group receiving 2500 ppm Zn. The effect on diarrhoea (reduced incidence of diarrhoea days and shorter duration of diarrhoea) improved as the amount of active ingredient in the animal feed product was increased.

### In Conclusion

Pigs and humans have anatomical and physiological similarities and the immune system of pigs is similar to that of humans. Therefore, the pig has been used for years as a valuable preclinical model for medical research on human products.

The results of the present study clearly illustrate that an animal feed product comprising the ingredient according to the present invention is a very interesting ingredient for improving the nutritional adsorption of the feed consumed and at the same time an inflammatory protection of the body was observed, since diarrhoea in piglets may be controlled too.

In addition to providing high antimicrobial effect, an increase in the intestinal villi height and the intestinal enterocyte height may also be provided, resulting in an increased internal surface area of the intestinal walls available for absorption of the nutrients from the digested feed as well as an increased nutritional adsorption.

### Example 2

An example similar to the example above was made, except that the animal feed was supplemented with a mixture of plant material (rapeseed meal, Brassica napus), and seaweed, Saccharina latisima/Ascophylum nodosum).

### Fermentation Procedure

The plant material and the seaweed were fermented via a 2-step solid state fermentation, using an inoculum consisting of three lactic-acid fermentative bacteria (Pediococcus acidilactici-DSM 16243, Pediococcus pentosaceus-DSM 12834, Lactobacillus plantarum-DSM 12837) to control the fermentation process, the fermentation process may be further described in WO 2014/206419, which is hereby incorporated by reference.

The addition of the inoculant controls the process by acidifying the blend to a pH 4.2 within the first 24 hours, providing an almost entire anaerobic process. The process runs for 11 days at 35° C. The fermented material is then dried in a spin flash dryer, with a temperature setting and pass-through-speed that preserves the viable bacteria and the microbial temperature-labile bioactive metabolites. The drying process is described in WO 2013/029632, which is hereby incorporated by reference.

### Feeding Procedure

This study was carried out at a commercial farm in Poland in 2018. In total, 666 eighteen-day-old piglets were tested under three feeding regimes. The following 3 different treatment groups (TG) were prepared:
TG 5: A commercial basal diet with no supplementation (negative control)
TG 6: A commercial basal diet, supplemented with 2500 ppm zinc oxide (positive control)
TG 7: A commercial basal diet, supplemented with 10% of the above fermented plant material and the seaweed

Animals were given the diets in separate groups, 10 days prior to weaning (at day 18 post farrowing) and until day 63 when piglets exited the nursing cage (at day 92 post farrowing). Piglets of each diet regime were housed in nursing pens holding an average of 48 animals per treatment. Each diet treatment was repeated 5 times and the control was repeated 4 times. The different pens were distributed and tagged accordingly in the nursing blocks at the farm.

None of the diets included growth promoter or antibiotics. Furthermore, piglets were not given administered antibiotics when presenting diarrhea at any point during the experiment. Animals that presented diarrhea were left in the pen untreated and allowed to recover by themselves, unless falling ill. Then they were removed from the experiment and treated by a veterinary.

The diets and fresh water were supplied ad libitum.

### Results and Discussion

In the same way as illustrated above in Example 1, with a feed supplemented with rapeseed alone, the results in the present Example 2 with the mixture of plant material (rapeseed meal, Brassica napus), and seaweed, Saccharina latisima/Ascophylum nodosum) also show to be a very interesting alternative and substitution to the addition of medicinal zinc, since a high antimicrobial activity in controlling diarrhoea in piglets is provided, as well as an increased intestinal villi height and intestinal enterocyte height are also be provided, resulting in an increased internal surface area of the intestinal walls available for absorption of the nutrients from the digested feed as well as an increased nutritional uptake resulting in increased weight gain.

Furthermore, the mucous membrane was higher in the TG 7 group (diet supplemented with mixture of plant material and seaweed) with significantly deeper intestinal crypts than in the TG 5 group (diet with no supplementation) and the TG 6 group (diet supplemented with zinc oxide).

Also the hindgut bacterial diversity was evaluated via 16S rRNA variable regions analysis and showed a significant decrease in the presence of the dominating Bacterioides fiber-associated Prevotella spp. whereas other other bacterial groups like, Lactobacillus spp, Ruminococcaceae spp., and the Clostridiales group increased significantly, compared to both the TG 5 group (diet with no supplementation) and the TG 6 group (diet supplemented with zinc oxide).

### Example 3

Effect of the particle size distribution.

The following 4 different treatment groups (TG) each comprising 25 piglets were provided with one of the following:
TG8: 10% of DM as prefermented dried rape seed
TG9: 8% of DM as prefermented dried rape seed +2% of DM as prefermented dried seaweed
TG10: 10% of DM as prefermented dried rape seed
TG11: 8% of DM as prefermented dried rape seed +2% of DM as prefermented dried seaweed

Ingredient TG 8 and TG 9 were mixed directly into the standard pre-weaner and starter diets.

Ingredients TG 10 and TG 11 were subjected to gentle grinding before they were mixed into the standard pre-weaner and starter diets. The resulting ground ingredients 7 and 8 has a particle size distribution where 50% (w/w) of the dried fermented ingredient has a particle size in the range of 0.01-0.5 mm, and 50% (w/w) of the dry fermented ingredient comprises a particle size in the range of 0.5-1.0 mm.

Feeding was done in the same way as described in Example 1.

### Results

The results demonstrate that the digestibility of the feed product was significantly improved in the treatment groups 10 and 11 compared to the treatment group 8 and 9.

This significant improvement may be caused by the smaller sized ingredients provided in treatment groups 10 and 11 where the digestive system in the pigs/piglet has a better access the to the material. Despite the treatment if ingredients 10 and 11, the results demonstrate a slight improvement in the nutritional uptake in treatment groups 10 and 11 compared to treatment groups 8 and 9.

The nutritional value per kg diet feed in treatment groups 10 and 11 compared to treatment groups 8 and 9 was calculated to 8% for treatment group 10 (compared to treatment group 8) and 10% for treatment group 11 (compared to treatment group 9).

### REFERENCES

WO 2008/006382
WO 2014/206419
PCT/EP2016/076952

1-14. (canceled)

15. A method for increasing microvilli height and/or enterocyte height in the intestine of a subject, by supplying to a subject an ingredient comprising one or more health-enhancing microorganism and:
(i) at least one fermented seaweed material;
(ii) at least one fermented plant material; or
(iii) a combination of at least one fermented seaweed material and at least one fermented plant material.

16. The method according to claim 15, wherein the ingredient comprises a pH 6 or below.

17. The method according to claim 15, wherein the ingredient comprises a pH 6 or below and wherein the drop in pH is provided by the fermentation process.

18. The method according to claim 15, wherein the ingredient is consisting essentially of one or more health-enhancing microorganism and:
(i) at least one fermented seaweed material;
(ii) at least one fermented plant material; or
(iii) a combination of at least one fermented seaweed material and at least one fermented plant material.

19. The method according to claim 15, wherein the one or more health-enhancing microorganism consists essentially of at least one lactic acid bacterial strain.

20. The method according to claim 15, wherein the fermented ingredient is a dried fermented ingredient.

21. The method according to claim 15, wherein the fermented ingredient is a dried fermented ingredient, and wherein a range of 20-80% (w/w) of the dry fermented ingredient has a particle size below 0.5 mm (preferably in the range of 0.01-0.5 mm), and wherein 20-80% (w/w) of the dry fermented ingredient comprises a particle size above 0.5 mm (preferably in the range of 0.5-1.0 mm).

22. The method according to claim 15, wherein the fermented ingredient is a dried fermented ingredient, and wherein the dried fermented ingredient comprises at least 2, preferably at least 3, even more preferably at least 4 of the following criteria:
a) 1-10% (w/w), such as about 5% (w/w), of the dry fermented ingredient has a particle size above 1.0 mm;
b) 45-55% (w/w), such as about 50% (w/w), of the dry fermented ingredient has a particle size between 0.5-1.0 mm;
c) 30-40% (w/w), such as about 50% (w/w), of the dry fermented ingredient has a particle size between 0.25-0.5 mm; and/or
5-15% (w/w), such as about 10% (w/w), of the dry fermented ingredient has a particle size below 0.25 mm.

23. The method according to claim 15, wherein the ingredient is used together with a basic food product to provide a human food product.

24. The method according to claim 23, wherein the ingredient represents in the range of 0.5-50% (w/w) of the human food product, such as in the range of 1-45% (w/w), e.g. in the range of 2-40% (w/w), such as in the range of 3-35% (w/w), e.g. in the range of 4-30% (w/w), such as in the range of 5-25% (w/w), e.g. in the range of 7.5-20% (w/w), such as in the range of 10-15% (w/w).

25. The method according to claim 23, wherein the basic food product is a dairy product, a cereal product, a prepared food product; a confectionary product, a sport nutritional product and a beverage fodder product suitable for human consumption.

26. A method for increasing the nutritional effect and/or the nutritional adsorption of a basic food product consumed by a human comprising supplying to a human, a human food product comprising a basic food product and an ingredient, wherein the ingredient comprising one or more health-enhancing microorganism and:
(i) at least one fermented seaweed material;
(ii) at least one fermented plant material; or
(iii) a combination of at least one fermented seaweed material and at least one fermented plant material.

27. The method according to claim 26, wherein the basic food product is a dairy product, a cereal product, a prepared food product; a confectionary product, a sport nutritional product and a beverage fodder product suitable for human consumption.

28. The method according to claim 26, wherein the ingredient represents in the range of 0.5-50% (w/w) of the human food product, such as in the range of 1-45% (w/w), e.g. in the range of 2-40% (w/w), such as in the range of 3-35% (w/w), e.g. in the range of 4-30% (w/w), such as in the range of 5-25% (w/w), e.g. in the range of 7.5-20% (w/w), such as in the range of 10-15% (w/w).

29. The method according to claim 26, wherein the human food product comprises the fibrous material originating from the plant material and/or the seaweed material.

30. The method according to claim 26, wherein the human food product comprises the starch material originating from the plant material and/or the seaweed material.
